(19) United States
(12) Patent Application Publication
(10) Pub. No.: US 2008/0177163 A1
(43) Pub. Date: Jul. 24, 2008

(54) VOLUMETRIC IMAGE FORMATION FROM OPTICAL SCANS OF BIOLOGICAL TISSUE WITH MULTIPLE APPLICATIONS INCLUDING DEEP BRAIN OXYGENATION LEVEL MONITORING

(75) Inventors: Shih-Ping Wang, Los Altos, CA (US); Xuefeng Cheng, Ontario (CA); Zengpin Yu, Palo Alto, CA (US)

Correspondence Address: COOPER & DUNHAM, LLP, 1185 AVENUE OF THE AMERICAS, NEW YORK, NY, 10036, US

(73) Assignee: O2 MedTech, Inc., Los Altos, CA (US)

(21) Appl. No.: 12/017,969

(22) Filed: Jan. 22, 2008

Related U.S. Application Data
(60) Provisional application No. 60/885,877, filed on Jan. 19, 2007.

(51) Int. Cl. A61B 5/1455

(52) U.S. Cl. 600/324; 600/340; 382/128

(57) ABSTRACT

Methods, systems, and related computer program products for non-invasive monitoring of a biological volume, such as a human brain, are described. In one preferred embodiment, each of a plurality of optical sources emits optical radiation into the biological volume each of a plurality of optical detectors detects optical radiation impinging thereupon from the biological volume. The optical measurements are processed to compute a requisite property value associated with each source-detector pair. For each source-detector pair, a volumetric basis region corresponding thereto is weighted by the requisite property value, the volumetric basis region being predetermined and representative of an estimated subvolume of the biological volume encountered by optical radiation emitted from that source and propagating to that detector. The weighted volumetric basis regions are accumulated into a volumetric cumulative array, and a display output is generated based at least in part on the volumetric cumulative array.

### CROSS-REFERENCE TO RELATED APPLICATIONS

This application claims the benefit of Provisional Application No. 60/885,877, filed Jan. 19, 2007, which is incorporated by reference herein. The subject matter of this application is related to the subject matter of US 2006/0015021A1, published on Jan. 19, 2006 (hereinafter “Cheng”), which is incorporated by reference herein.

### FIELD

This patent specification relates to patient monitoring, including the volumetric imaging of chromophore concentrations or other properties of biological tissue using information acquired from non-invasive optical scans thereof, such as near infrared optical scans. Although applicable in a wide variety of contexts, one particularly advantageous use is for deep brain oxygenation level monitoring.

### BACKGROUND AND SUMMARY

The use of near-infrared light as a basis for the measurement of biological properties or conditions in living tissue is particularly appealing because of its relative safety as compared, for example, to the use of ionizing radiation. Various techniques have been proposed for non-invasive near-infrared spectroscopy of biological tissue. Generally speaking, these techniques are directed to detecting the presence and/or measuring the concentrations of one or more chromophores in the biological tissue, such as blood hemoglobin in oxygenated (HbO) and deoxygenated (Hb) states.

However, the types of infrared measurements that are sufficiently developed for practical and everyday medical use are basically directed to “aggregate” or “average” measurement of a biological property over relatively large areas of tissue. It would be desirable to provide for non-invasive infrared measurement of biological tissue sufficiently localized such that actual spatial maps of the biological property or condition are provided that can be expressed as viewable medical images, or medical images suitable for further processing or other uses, while maintaining the safety, low cost, and relative simplicity associated with current “aggregate” or “average” infrared measurement devices.

One exemplary need for fast, safe volumetric imaging of chromophore concentrations in biological tissue, particularly oxygenated hemoglobin levels for the deep human brain, arises in the context of the millions of surgical procedures performed under general anesthesia every year. A real-time, constantly updated three-dimensional map of blood oxygenation levels in the deep brain (i.e., about 4-8 centimeters inward from the skin surface) would allow the anesthesiologist to readily detect anomalies that can lead to permanent (or reversible) brain damage or death of the patient.

Hospital emergency rooms represent another real-world need for fast, safe volumetric imaging of chromophore concentrations, in particular oxygenated hemoglobin levels for the deep human brain. It is often the case, for example, that a patient will arrive in the emergency room in a disoriented and confused state. This condition can have a variety of causes ranging from relatively benign causes, such as drug side effects, to critical and time-sensitive causes, such as stroke or hematoma. It would be desirable to quickly, safely, and inexpensively provide a three-dimensional blood oxygenation map of the patient's brain so that the emergency room physician can (a) take prompt action if the deep brain blood oxygenation map is indicative of a stroke, hematoma, etc., while at the same time (b) prevent excessive equipment and personnel costs if the blood oxygenation map is normal and therefore indicative of a more benign cause for the patient's condition.

Additional issues that may arise in patient monitoring contexts include integrating the hardware and/or software associated with non-invasive three-dimensional mapping of oxygenation levels with more conventional non-invasive monitoring functionalities such as pulse oximetry functionalities. It is to be appreciated that the scope of the preferred embodiments infra is not limited to addressing the above-described issues, and that one or more of the preferred embodiments infra may be directed to resolving other issues that may arise in non-invasive patient monitoring, such as the avoidance of unnecessary redundancies in acquiring multiple status metrics such as cerebral tissue oxygenation metrics and arterial blood oxygenation metrics.

According to one preferred embodiment, a method, system, and related computer program products are provided for volumetric imaging of the chromophores in biological tissue, such as volumetric imaging of blood oxygenation levels in the deep brain, using optical scans thereof such as infrared optical scans. Measurements are acquired from a population of detectors spatially arranged around at least a portion of the biological volume of interest, the detectors measuring optical radiation emitted from a plurality of sources also spatially arranged around that portion. At least one optical radiation measurement is acquired for each source-detector pair. From these optical radiation measurements, a three-dimensional volumetric representation of a requisite property value for the biological volume of interest is computed. Any of a variety of two dimensional and three dimensional display techniques, such as cine-style viewing of two-dimensional images, maximum intensity or other projections or other mappings of 3-dimensional images, various grayscale or color codings, etc., can then be used to display a volumetric image to a physician or other viewer, each voxel of the volumetric image corresponding to the value of the requisite property thereat and/or other metrics that can be derived from the value of the requisite property thereat. In addition, further processing may be applied to such images to present the information in different ways or to extract other information from them or to combine information from such images with other information.

For one preferred embodiment, for each source-detector pair there is a predetermined volumetric basis region representing a subvolume of tissue encountered by optical radiation emitted from that source and propagating to that detector. The volumetric basis region comprises both an overall shape and a weighting profile, the weighting profile reflecting the fact that certain voxels within that shape are traversed more often than others for radiation traveling from that particular source to that particular detector. The volumetric basis region is predetermined by statistical computation, measurement, or other predefinition or computational technique using a model or other estimate of the requisite property of the biological volume. For each source-detector pair, the at least one optical radiation measurement is processed to produce a requisite property value for that source-detector pair. A backprojection and reconstruction algorithm is then applied in which the volumetric basis region for each source-detector pair is weighted by the requisite property value for that source-detector pair, and all of the weighted volumetric basis regions are summed together on a voxelwise basis into a volumetric cumulative array. The volumetric cumulative array is then filtered (e.g., using a high-pass filter) and/or normalized to result in the three-dimensional volumetric representation of the requisite property value for the biological volume of interest.

### BRIEF DESCRIPTION OF THE DRAWINGS

FIG. 1 illustrates a conceptual view of a first plurality of volumetric basis regions extending between a first source and a respective plurality of detectors;

FIG. 2 illustrates a conceptual view of a second plurality of volumetric basis regions extending between a second source and a respective plurality of detectors;

FIG. 3 illustrates a simplified Cartesian map of volumetric basis regions topologically equivalent to the volumetric basis regions of FIG. 1;

FIG. 4 illustrates an example of backprojection, accumulation, filtering, and normalizing according to a preferred embodiment, using simplified Cartesian maps for purposes of illustration;

FIG. 5 illustrates an output display according to a preferred embodiment;

FIG. 6 illustrates a source-detector network of a non-invasive scanning device according to a preferred embodiment;

FIG. 7A illustrates a front view of a patient with a wearable source-detector array according to a preferred embodiment;

FIG. 7B illustrates a conceptual cross-sectional view of penetration paths among respective pairings of sources and detectors for a portion of the source-detector array of FIG. 7A;

FIG. 8 illustrates a simplified example of a brain mapping table according to a preferred embodiment;

FIG. 9 illustrates a block diagram of a three-dimensional brain scanner device according to a preferred embodiment;

FIG. 10 illustrates a system connection diagram of a three-dimensional brain scanner device according to a preferred embodiment;

FIG. 11 illustrates a system block diagram corresponding to the three-dimensional brain scanner device of FIG. 10;

FIG. 12 illustrates a non-invasive scanning apparatus and an associated user display according to a preferred embodiment;

FIG. 13 illustrates computation of arterial oxygen saturation as an adjunct to measuring cerebral tissue oxygen saturation according to a preferred embodiment; and

FIG. 14 illustrates a non-invasive scanning apparatus and an associated user display according to a preferred embodiment.

### DETAILED DESCRIPTION

Hemoglobin, the molecule that carries oxygen in the blood, can exist an oxygenated state, which is designated herein as HbO, and a deoxygenated state, designated herein as Hb. Total hemoglobin, which can be designated as “total Hb” or “HbT”, refers to the collection of the oxygenated and deoxygenated states of hemoglobin (total Hb=HbT=HbO+Hb). Total hemoglobin concentration, which is designated herein by the symbol [HbT], refers to the amount of hemoglobin per unit of blood, and is often expressed in grams per deciliter (g/dl). Similarly, oxygenated hemoglobin concentration, which is designated herein by the symbol [HbO], refers to the amount of oxygenated hemoglobin per unit of blood, and deoxygenated hemoglobin concentration, which is designated herein by the symbol [Hb], refers to the amount of deoxygenated hemoglobin per unit of blood, and both quantities can likewise be expressed in grams per deciliter (g/dl).

Oxygen saturation refers to the fraction (which can be stated as a decimal between zero and one or equivalently as a percentage) of total hemoglobin that is oxygenated, and can be computed as [HbO]/[HbT]=[HbO]/([HbO]+[Hb]). Under normal conditions, arterial oxygen saturation, which is designated herein as SaO2, is often in the neighborhood of 95 percent, while venous oxygen saturation, which is designated herein as SvO2, is often in the neighborhood of 70 percent. Cerebral tissue encompasses a mixture of smaller artery branches, arterioles, capillaries, venules, smaller vein branches, etc. Cerebral tissue oxygen saturation, which is designated herein as SctO2, can vary on a localized basis, and a volumetric mapping of SctO2 could provide crucial information for clinical and medical applications, such as cortex perfusion monitoring, diagnosis of hematoma, stroke, organ function monitoring, etc.

A particular preferred embodiment is now described with reference to oxygenated hemoglobin (HbO) concentrations (and related quantities) in the deep brain using near-infrared optical radiation. It is to be appreciated that although the use of optical radiation in the range of 600 nm-900 nm, which is particularly advantageous for HbO concentration (and related quantities) measurement, the scope of the preferred embodiments is not so limited. In other preferred embodiments, as may be dictated by the particular type of chromophore being detected and/or other factors, optical radiation from 100 nm-5000 nm or from 300 nm-3000 nm may be used. In still other embodiments, the optical radiation may be in the broader “near-infrared” range of 500 nm-2500 nm. Thus, the use of the terms near-infrared or infrared” in the examples herein should not be construed as limiting the scope of the present teachings to that particular range of optical frequencies.

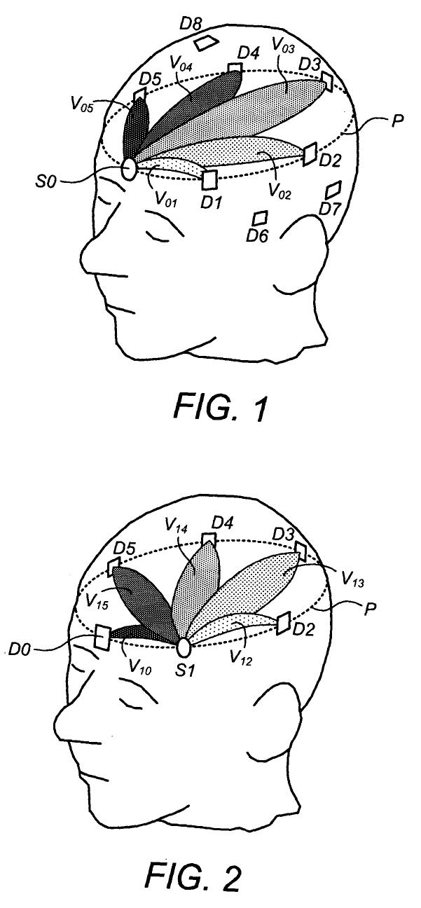

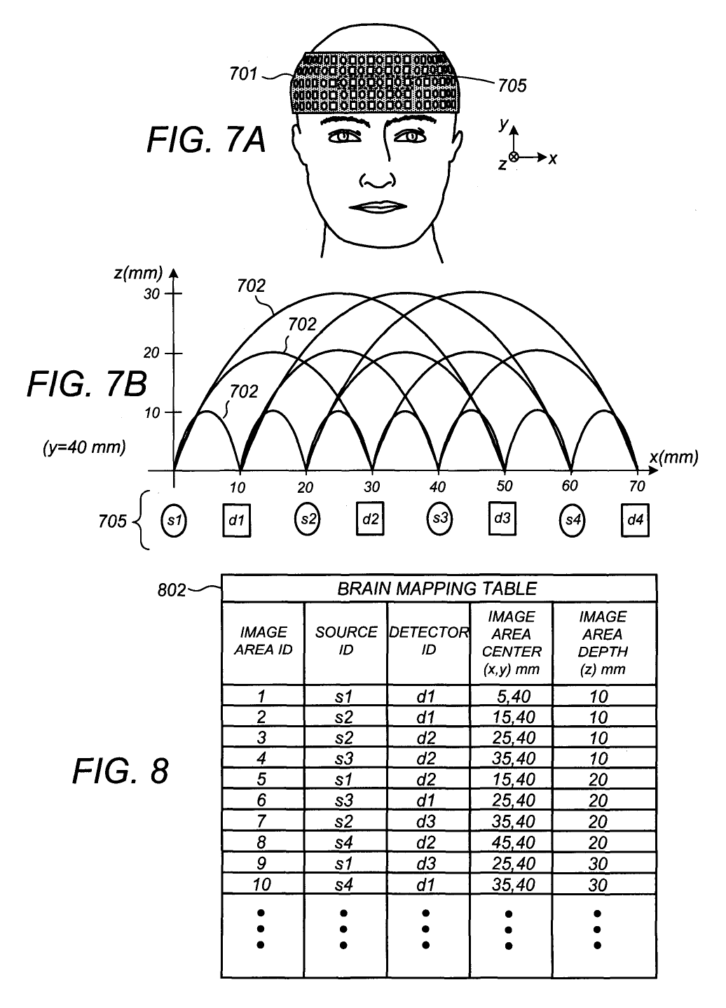

FIG. 1 illustrates a conceptual perspective view of a patient in which a an infrared source S0 is emitting infrared radiation while a plurality of detectors D1-D5 are detecting infrared radiation. For purposes of clarity, only a single infrared source S0 is illustrated in FIG. 1, while more generally, according to a preferred embodiment, a plurality of infrared sources and a plurality of infrared detectors are positioned against the skin of the patient's head, preferably distributed entirely around the patient's head, with sources and detectors being paired together in a spatially dispersed arrangement. Although FIG. 1 illustrates for clarity only a single source or detector at any particular node “n”, each of those nodes preferably contains both a source Sn and a detector Dn. The sources and detectors can be similar to those described in Cheng, supra. The detectors can optionally comprise photomultipliers with fiber couplings for very high sensitivity. The patient's head can or should be shaved or otherwise treated to facilitate good optical coupling between the source and detectors. Preferably, an elastic headband-like or hat-like apparatus is provided in which the sources and detectors are pre-installed (see FIG. 7A, infra) and an optically translucent adhesive is used to secure the sources and detectors to, or close to, the skin surface to facilitate optical coupling.

The sources and detectors preferably are driven such that only a single source emits at any given moment, with multiple detectors receiving the emitted radiation energies, e.g., photons, after they have propagated and scattered or otherwise modulated through the tissue volume to their respective locations. Thus, FIG. 1 illustrates a conceptual example of a particular point in time when the source S0 is emitting and D1-D5 are detecting. Between the source S0 and each of the detectors D1, D2, D3, D4, and D5 there arises a respective volumetric basis region, designated in FIG. 1 as V₀₁, V₀₂, V₀₃, V₀₄, and V₀₅.

FIG. 2 illustrates a conceptual example of the patient and source-detector pairs from FIG. 1 at a different point in time when a different source S1 is emitting, while detectors D0 and D2-D5 are detecting. Each volumetric basis region Vₙₘ extending between source Sn and detector Dm comprises the voxels in the tissue volume that would be traversed, in a “model” or “typical” version of that tissue volume, by a population of optical energies, e.g., photons, originating at source Sn and arriving at detector Dm, each member voxel being weighted relative to the other member voxels according to what statistical percentage of those photons would actually pass through that voxel on their way from that source Sn to that detector Dm. By way of comparison to probability clouds from quantum physics, volumetric basis regions can take on particular shapes when viewed in isosurface, isoline, threshold, etc. For the head/deep brain, typical volumetric basis regions will be (a) volumetrically ellipsoidal or “cigar-shaped” when the source and detector are on opposite sides of the head, and will tend toward (b) “banana” shapes for source-detector pairs on the same side of the head.

Although shown in a single plane “P” for clarity of description in FIG. 1, the sources and detectors will generally be interspersed at many different locations in many different planes (see, e.g. detector locations D6, D7, and D8 in FIG. 1), so that points in both shallow and deep-brain areas are each covered by several volumetric basis regions. In one preferred embodiment, the shape and weighting factors for the volumetric basis regions can be computed by (a) estimating the requisite property to be measured by the optical, e.g., infrared, radiation for a “model” head, and then (b) for each source-detector pair, using a Monte Carlo simulation method and to statistically track the voxels traversed. Monte Carlo simulation methods are known in the art and are quite popular in medical physics; see, for example, Boone, et. al., “Monte Carlo Simulation of the Scattered Radiation Distribution in Diagnostic Radiology,” Med. Phys. 15 (5), pp. 713-20 (September/October 1988), which is incorporated by reference herein.

In one embodiment presented by way of example and not by way of limitation, there may be 40 sources S0, S1, . . . S39 and 100 detectors D0, D1, . . . D99. Only one of the sources Sn is actively emitting during any particular time interval, while all of the detectors Dm are active at all time intervals, such that at least one measurement (which can be a long vector of measurements or just one scalar value depending on the property to be measured) is acquired for each source-detector pair. For this example, there are 4000 source-detector pairs and therefore 4000 volumetric basis regions, and if the volumetric basis regions are hypothetically remapped spatially into Cartesian coordinates, this would correspond to a 20×20×10 resolution (corresponding to voxel sizes of roughly between about 0.5 cm×0.5 cm×0.5 cm and 1 cm×1 cm×1 cm for the head) which, although not precisely indicative, is generally illustrative of the resolutions that can be readily achieved for deep brain volumetric imaging according to this preferred embodiment, and even better resolutions are achieved using more sources and/or detectors.

Although the preferred embodiments are generally applicable to a variety of different properties that can be determined for volumetric basis regions for the source-detector pairs, it has been found especially advantageous to use the methods of Cheng, supra, as the basis for computing requisite property values that are then further processed according to the volume imaging methods described herein. Thus, for one preferred embodiment, the process starts with the source S0 providing an optical signal having different wavelength and modulation frequencies for different time intervals. For a first interval, the wavelength of the optical signal is 690 nm, and the intensity is modulated (e.g., by a 50% duty cycle on/off square wave) at a frequency of 100 MHz. For a second interval, the wavelength remains at 690 nm and the modulation frequency is changed to 300 MHz. For a third interval, the wavelength remains at 690 nm and the modulation frequency is changed to 800 MHz. The wavelength is then changed to 780 nm, and the above progression of different modulation frequencies is repeated. Finally, the wavelength is changed to 830 nm, and the above progression of different modulation frequencies is again repeated. In summary, the optical signal provided by the source S0 comprises nine different combinations of wavelength and modulation frequency provided during nine different sub-intervals. During these nine different sub-intervals, the remaining sources S1-S39 are silent, and the detectors D1 through D99 are receiving. The source S0 is then silenced, and the entire sequence is repeated for source S1, with detectors D0 and D2-D99 receiving, and so on until all 40 sources have sent their optical signals. The entire process then repeats on a continuing basis throughout the medical procedure or monitoring interval, with the computations and associated volumetric images being updated in real time as the measurements are taken. Different wavelengths, number of wavelengths, modulation frequencies, number of modulation frequencies, and firing sequences are also within the scope of the preferred embodiments. For example, another suitable set of modulation frequencies comprises 100 MHz, 120 MHz, and 140 MHz. Alternative sequencing is possible, such as energizing the sources in sequence at one frequency, then changing to another frequency, energizing some of the sources and then others at a given frequency, etc.

Referring now to any particular source-detector pair, the detector detects a received version of the optical signal, and a phase shift that occurs in association with the intensity modulation signal is extracted from that received version. This is performed for each of the nine different combinations of wavelength and modulation frequency for that source-detector pair. These phase signals are then processed to compute a requisite property value that is applicable to that source-detector pair. As used herein, requisite property value refers generally to one or more computed metrics that are directly or indirectly indicative of a chromophore concentration or saturation metric, or from which such concentration or metric can be computed. Preferably, the requisite property value is selected to be a metric that is amenable, as can evidenced by direct analysis and/or empirically, to superposition or accumulation (including filtered superposition or accumulation) with like metrics from other source-detector pairs to arrive at a useful overall value thereof for the tissue of interest at which the superposition or accumulation occurs. It has been found that one particularly effective requisite property value is the absorption coefficient μₐ for each wavelength of the optical signal that was applied. In addition to the fact that each absorption coefficient μₐ for each wavelength is particularly amenable to filtered superposition or accumulation across multiple source-detector pairs, the absorption coefficients μₐ at the different wavelengths at any particular voxel can be used to directly compute or estimate the absolute chromophore concentrations [Hb] and [HbO] and absolute oxygen saturation metric at that voxel.

According to a preferred embodiment, again referring to the particular source-detector pair and the related measurement comprising the multiple phase readings, the mathematical relationships described at [0039]-[0040] of Cheng, supra, are used to compute the absorption coefficient μₐ for each wavelength of the optical signal that was applied, designated herein as absorption coefficient μₐ^{λi}. For each absorption coefficient μₐ^{λi} that has been computed across all source-detector pairs, a volumetric map of that absorption coefficient μₐ^{λi} is computed by weighting each volumetric basis region with the absorption coefficient μₐ^{λi} computed for that source-detector pair (i.e., backprojecting μₐ^{λi} into the cerebral tissue volume), accumulating all of the weighted volumetric basis regions on a voxelwise basis, and then filtering and normalizing the result. For the above example in which there are 40 sources and 100 detectors (4000 source-detector pairs), the backprojection and accumulation process comprises 4000 weightings 4000 voxelwise accumulations of volumetric basis regions (the “cigars” or “bananas”). The volumetric cumulative array is then filtered (e.g., using a high-pass filter) according to known backprojection/reconstruction methods (e.g., the spatial and/or frequency domain reconstruction methods used in CT and MRI image reconstruction) and preferably normalized to arrive at the desired volumetric image of the oxygenated hemoglobin concentration.

Using an example in which the three wavelengths 690 nm, 780 nm, and 830 nm are used, there results three volumetric maps after the above process is completed, which can alternatively be considered, of course, as a single volumetric map in which each voxel takes on three values μₐ⁶⁹⁰, μₐ⁷⁸⁰, and μₐ ⁸³⁰. According to a preferred embodiment, a relationship such as set forth in Cheng, supra at [0045] is then used to compute, for each voxel in the brain tissue volume of interest, the absolute chromophore concentrations [HbO] and [Hb] from the computed values of μₐ⁶⁹⁰, μₐ⁷⁸⁰, and μₐ⁸³⁰, from which is directly yielded the absolute oxygen saturation SctO2 for each voxel in the brain tissue volume of interest. The three-dimensional SctO2 volume can then be displayed using any of a variety of two dimensional and three dimensional display techniques, such as cine-style viewing of two-dimensional images, maximum intensity projection of 3-dimensional images, various grayscale or color codings, etc.

It is to be appreciated that, although each of the absorption coefficients μₐ^{λi} is particularly useful as the requisite property value that is backprojected and reconstructed in accordance with a preferred embodiment, the scope of the present teachings is not so limited. For example, in another preferred embodiments, the requisite value property value that is backprojected and reconstructed can comprise the scattering coefficient(s). In still other preferred embodiments, the requisite property value that is backprojected and reconstructed can comprise one or more of [HbO], [Hb], [HbT], SctO2, or combinations thereof, or any other result that can be determined on a per source-detector-pair basis. Moreover, although displays of three-dimensional SctO2 maps are particularly useful, it is within the scope of the preferred embodiments to display, alternatively or in conjunction therewith, three-dimensional maps of the one or more of one or more of the [HbO], [Hb], and [HbT] results.

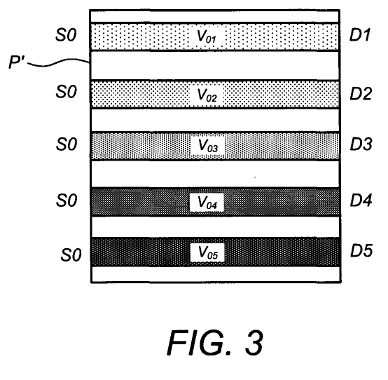

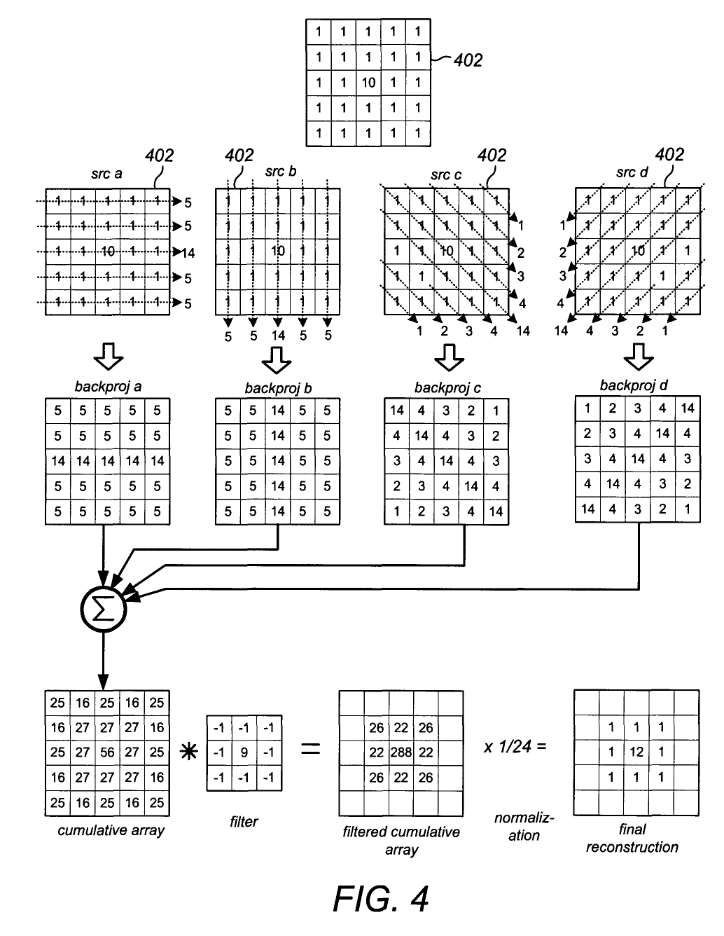

FIGS. 3-4 illustrate a conceptual example of backprojection reconstruction to compute a three-dimensional brain map according to a preferred embodiment, with mappings made (using methods known in the art) into Cartesian coordinates for clarity of disclosure. FIG. 3 illustrates a two-dimensional map P′ using the optical source S0 and detectors D1-D5 from FIG. 1, supra, which is topologically equivalent to the “fan beam” of FIG. 1 with each ellipsoidal (banana or cigar shaped) volumetric basis region now in the form of a pencil-like parallel beam.

FIG. 4 illustrates a simplified conceptual example of optical scanning, measurement, backprojection and reconstruction proceeding according to a preferred embodiment, again for a simplified Cartesian example, for a brain having a very simple attenuation (e.g., absorption) map 402. Four different scans from four different directions are made using four respective sources a, b, c, and d and between five and nine associated detectors. The reading from any one of the detectors is a sum of the attenuation coefficients in the light beam path, which can be done because the light beam attenuation is proportional to the log of the detector reading (of the transmitted signal), that is, log T=Sum(−μt), in which μ is the attenuation coefficient and t the thickness. In the real world, t is a known value, since we know or can measure or estimate the distance between the source and the detector. The ellipsoid, as a first approximation, could be replaced by a straight line or a curved line. The shape of the ellipsoid could be readily determined using, for example, Monte Carlo simulation, as discussed supra. The shape of the ellipsoid would determine the size of the voxels. That is, the voxels will be larger in deep brain and smaller near the outer surface of the brain. Referring again to FIG. 4, measurements resulting from the time-distinct application of sources a, b, c, and d are then backprojected, and the resultant backprojections are summed into a cumulative array. Filtering and normalization is then performed on the cumulative array to produce the final reconstruction. As expected, the final reconstruction is a very close approximation to the original attenuation map 402. For one or more preferred embodiments, known methods used in the reconstruction of x-ray CT images from x-ray CT scans may be used. The use of a variety of filters and normalization schemes would be apparent to a person skilled in the art in view of the present disclosure (a simplified high-pass filter and normalization by a predetermined constant are used in the example of FIG. 4) and are within the scope of the preferred embodiments. While FIG. 4 illustrates a particular example of spatial domain backprojection followed by spatial domain filtering and normalization, it should be clear that this is just a simplified example of spatial backprojection image reconstruction, and that there are other varieties of such reconstruction known in the art, e.g., in CT (computerized tomography) image reconstruction. Further, the term reconstruction as used in this patent specification also encompasses image reconstruction in the time domain (Fourier Transform or Fourier Series) image reconstruction as commonly used in contemporary CT, MRI (magnetic resonance imaging) and tomosynthesis.

For one preferred embodiment, the source-detector pairs are distributed around the head in a three-dimensional (non-coplanar) arrangement, the volumetric basis regions are three-dimensional, each source-detector measurement is processed to produce the absorption coefficients μₐ^{λi}, the backprojection/reconstruction for each absorption coefficient μₐ^{λi} is performed in three dimensions, and the three-dimensional maps are converted to a corresponding three-dimensional map of SctO2 on a voxelwise basis. For an alternative preferred embodiment, the source-detector pairs are distributed around the head in a two-dimensional (coplanar) arrangement (for example, limited to the plane P in FIG. 1), the volumetric basis regions are collapsed into two-dimensional collapsed volumetric basis regions, each source-detector measurement is processed to produce absorption coefficients μₐ^{λi} that are applicable to the associated two-dimensional collapsed volumetric basis region, the backprojection/reconstruction for each absorption coefficient μₐ^{λi} is performed in two dimensions, and the two-dimensional maps are converted to a corresponding two-dimensional map of SctO2 on a voxelwise basis.

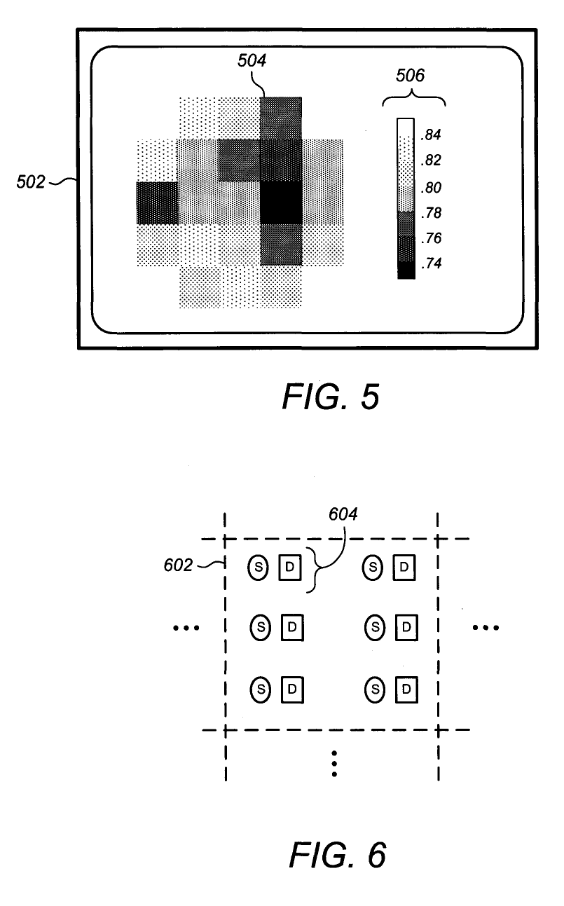

FIG. 5 illustrates a display 502 according to a preferred embodiment for presenting results of the above-described three-dimensional brain mapping process to a clinician or other user. The display 502 illustrates a two-dimensional image 504 representing SctO2 in grayscale-coded or color-coded format according to a key/legend 506. FIG. 6 illustrates an example of a source-detector network 602 forming all or a portion of a non-invasive scanning device according to a preferred embodiment, comprising a population of measurement units 604. As used herein, measurement unit refers to a physically related, e.g., adjacent, source-detector pair.

FIG. 7A illustrates a front view of a patient with a handband-like wearable source-detector array 701 according to a preferred embodiment, including a segment 705 thereon comprising a row of measurement units. FIG. 7B illustrates a conceptual cross-sectional view of penetration paths 702 among respective pairings of sources and detectors in the segment 705. The penetration paths 702 correspond roughly to a conceptual “center line” of the volumetric basis region for each source-detector pair.

FIG. 8 illustrates a simplified example of a brain mapping table 802, which can be implemented in hardware, firmware, or software as part of a processing device optimized for real time computation of a three-dimensional brain map of SctO2 (or other related property) according to a preferred embodiment. In accordance with parameters stored in the brain mapping table 802, each source-detector pair (e.g., s1d1, s2d1, s2d2, s4d2, etc.) maps into a predetermined portion (more specifically, its volumetric basis region) of a three-dimensional data volume representing the tissue volume of interest. Phase measurements for that source-detector pair are processed into the requisite property values as they are received, and the requisite property values are stored in that volumetric basis region of the three-dimensional data volume as they are computed, as weighted according to the volumetric basis region weights and, optionally, filtered/normalized according to the reconstruction filtering/normalizing algorithm. The three-dimensional data volume thus represents a three-dimensional map of the requisite property that is stored and updated on a continuing basis as scans are taken. It is to be appreciated that there are two distinct sets of weights discussed above, the first set of weights being the requisite property values (one requisite property value per volumetric basis region to weight that volumetric basis region before accumulation), the second being the fixed, precomputed or otherwise computed or estimated relative weight of each voxel of a volumetric basis region relative to the other voxels that serves to “shape” that volumetric basis region.

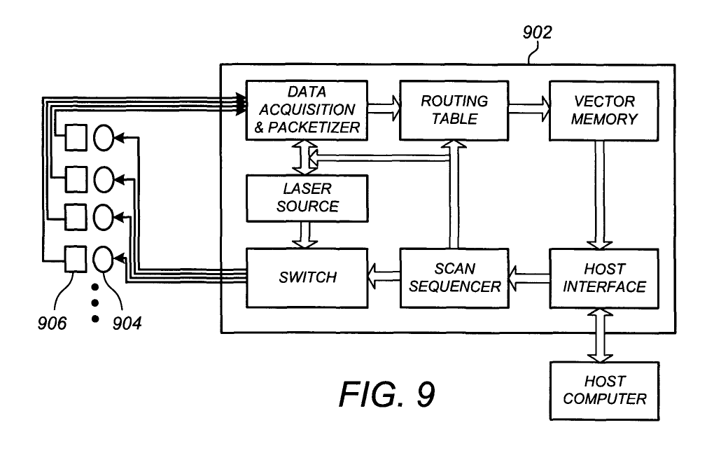

FIG. 9 illustrates a block diagram of a three-dimensional brain scanner device based on optical switching according to a preferred embodiment. Sources 904 and detectors 906 are coupled to a hardware interface 902, which in turn is coupled to a host computer. A host interface downloads brain scan parameters and commands, uploads raw photodetector vector data, and performs system diagnostics. A scan sequencer manages the laser light source transmit power, manages scan timing for each source-detector pair, provides header information for the packetizer (e.g., scan ID, photodetector ID, number of samples), and manages scan transmit and receive timing. A laser source provides optical radiation energies, e.g., photons, that travel through an optical switch and fiber optic cables to a selected light source, the photons entering into the skin through an optic interface window formed in a light insulating layer of the wearable device. The light insulating layer comprises a material such as bio-compatible rubber making optical contact with the patient's skin. A data acquisition and packetizer unit provides multiplexing for received analog signals, generates adjustable sampling frequencies and data lengths for analog to digital conversion, and generates data packets with data headers based on information provided by the scan sequencer. A routing table provides a starting location of a vector memory for storing incoming data packets, and organizes vector memory in 3D format for image reconstruction. A vector memory stores data packets from the data acquisition unit and stores vector data in 3D format.

In operation, the host computer downloads the scan pattern into the scan sequencer. For each scan vector, there is a scan command associated with it, which can be defined as following: Scan ID (used as a data header for vector data packet), Laser Power ID (used to select laser transmit power), Sensor ID (used to select a receive channel from analog signal multiplexer), Switch ID (used to open an optic switch channel, enabling laser light to pass to a light source via fiber optic cable), and Routing ID (used to index into routing table to store acquired data into vector memory). The scan sequencer issues a start command, and the scan control word for one source-detector pair is sent to corresponding hardware control circuits. In particular, the Scan ID and Sensor ID are sent to the data acquisition and packetizer unit, the Laser Power ID is sent to the laser control circuit, the Switch ID is sent to the switch unit, and the routing ID is sent to routing control circuits. This sequence continues until all pre-loaded scan commands have been executed.

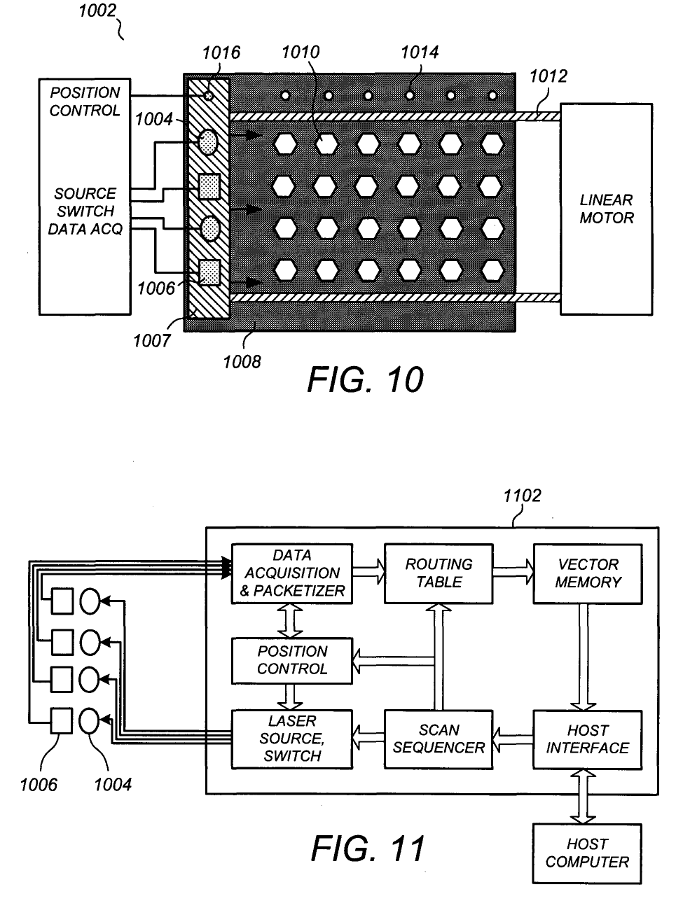

FIGS. 10-11 illustrate a system connection diagram and a system block diagram, respectively, of a three-dimensional brain scanner device 1002 based on motor control according to a preferred embodiment. Sources 1004 and detectors 1006 are driven by a hardware interface 1102, which in turn is coupled to a host computer. Mounted on a movable dielectric film flex circuit assembly 1007 are photodetectors 1006, as well as sources 1004 that receive light from a laser source through a switch and respective fiber optic cables. The assembly 1007 is connected to a linear motor via a pair of belts/tracks 1012. An optically opaque contact structure 1008 includes a bio-compatible rubber material for making contact with the skin. Formed into the contact structure 1008 are discrete optical interface holes 1010 for allowing light to pass in and out of the skin at predetermined locations, as well as fiducial holes 1014 in alignment therewith for detection by a position sensor 1016. Preferably, preamplifiers are placed on the flex circuit of the assembly 1007 along with the photodetectors. Proper grounding plants are laid out on both sides of the flex circuits to provide electromagnetic interference shielding. In one preferred embodiment, signal conditioners, signal filters, and analog-to-digital converter(s) are also placed on the flex circuit of the assembly 1007 to improve system performance.

In operation, the linear motor makes a stop based on input from the position sensor 1016, the optic interface holes 1010 then providing the paths for the laser light to penetrate into the patient skin and for propagated light to travel to the photodetectors 1006. A motion control assembly, illustrated as a position control box in FIG. 11, controls the linear motor to move the assembly 1007, stops the linear motor when a defined position is reached, and provides position information. A scan sequencer manages the operation of the motion control assembly, provides timing control for laser light transitions and photodetector data acquisition, and organizes vector data for storage in the vector memory. During system reset, the assembly 1007 is moved to a pre-defined system origin. The scan sequencer starts to command one source-detector pair to emit and receive. Vector data is stored in the vector memory based on the position of assembly 1007 and the order of source-detector pair operation. The scan continues until all pre-defined source-detector pairs on assembly 1007 have operated. The assembly 1007 is moved to the next position defined by the next fiducial hole 1014, and the process is repeated until the end of the scan is reached.

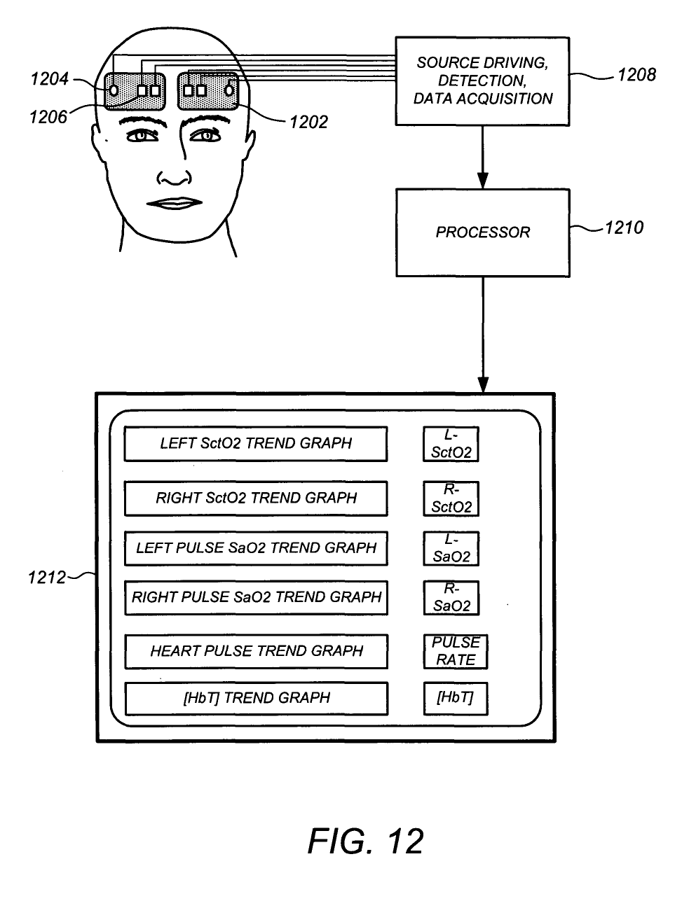

FIG. 12 illustrates a non-invasive scanning apparatus and an associated user display 1212 according to a preferred embodiment in which arterial oxygen saturation (SaO2) is monitored as an adjunct functionality of regional cerebral oxygen saturation (SctO2) monitoring, in contrast to using separate pulse oximeter hardware. Forehead patches 1202 mounted to the forehead each contain a source 1204 and detectors 1206, and are driven by a hardware interface 1208. The hardware interface 1208 is coupled to a processor 1210 that processes measurements from the forehead patches and provides information for the user display 1212. Preferably, the user display 1212 simultaneously displays the following: left and right SctO2 trend graphs, left and right SctO2 current levels, left and right SaO2 trend graphs, left and right SaO2 current levels, a heart pulse trend graph, a current pulse rate, an absolute [HbT] trend graph, and an absolute [HbT] current level. Trend graphs preferably comprise results over a time period sufficient to suggest a trend in the displayed results.

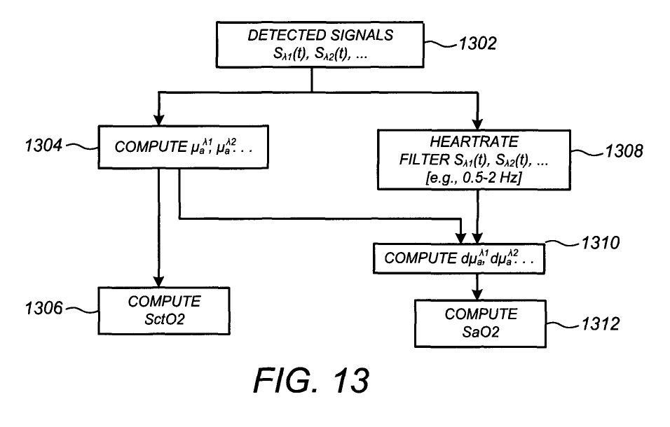

FIG. 13 illustrates computation of SaO2 as an adjunct to measuring SctO2 according to a preferred embodiment. At step 1302, time signals S_{λ1}(t), S_{λ2}(t), . . . corresponding to the readings from detectors 1206 for at least one wavelength are received. For one preferred embodiment, the time signals S_{λ1}(t), S_{λ2}(t), . . . can be intensity signals received in a continuous-wave (CW) implementation. In another embodiment, the time signals S_{λ1}(t), S_{λ2}(t), . . . can be phase signals extracted in a phase-modulated spectrophotometry (PMS) implementation. At step 1304, a first set of absorption coefficients μₐ^{λi} as may be used in computing SctO2 are computed, and at step 1306 the output value of SctO2 is computed using the relationship of Cheng, supra, at [0045] (via a computation for the chromophore concentrations [Hb] and [HbO]). For a CW implementation, variations in signal intensity that occur at the rate of the patient's pulse can be associated with arterial absorption. At step 1308, the time signals S_{λ1}(t), S_{λ2}(t), . . . are filtered in time to extract components thereof that vary with the patient's pulse, which can be performed using a low-pass (e.g., 0.5-2 Hz) heart-rate filter. In other preferred embodiments, lock-in amplification and phase sensitive detection can be used. At step 1310, a second set of absorption coefficients dμₐ^{λi} are computed based upon the heart-rate components extracted at step 1308 and the first set of absorption coefficients. At step 1312, the output value of SaO2 is determined from dμₐ^{λi} also using the relationship of Cheng, supra, at [0045]. Analogous methods can be used for PMS implementations.

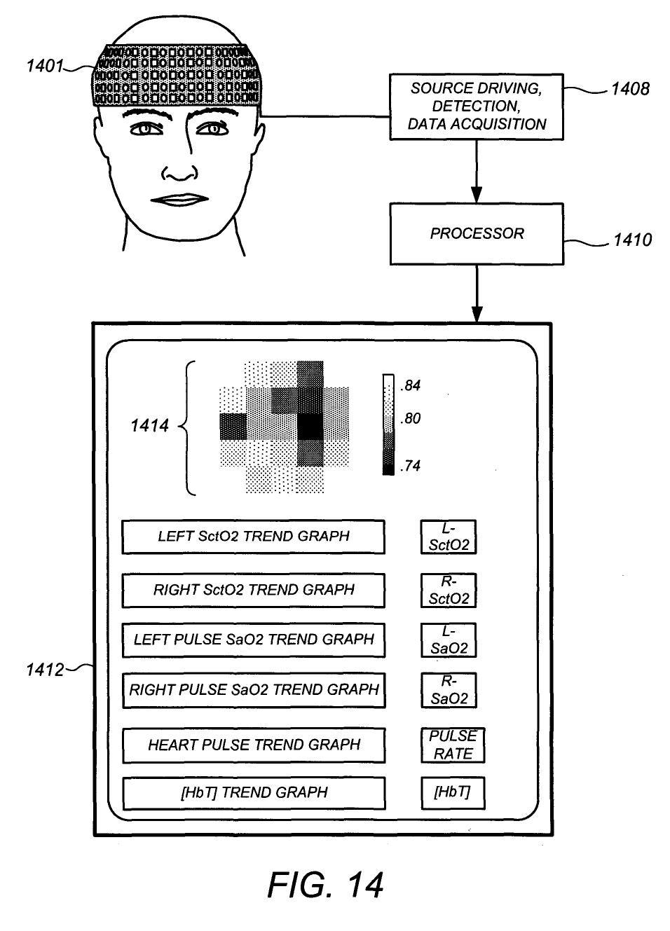

FIG. 14 illustrates a non-invasive scanning apparatus and an associated user display 1412 according to a preferred embodiment in which arterial oxygen saturation (SaO2) is monitored as an adjunct functionality of three-dimensional volumetric brain mapping of cerebral oxygen saturation (SctO2) monitoring, which is also in contrast to using separate pulse oximeter hardware for the SaO2 measurement. A wearable headband-like device 1401 comprising a population of source-detector measurement units is provided that preferably encompasses most or all of the head as shown. The sources and detectors are driven by a hardware interface 1408. The hardware interface 1408 is coupled to a processor 1410 that processes measurements from the wearable headband-like device 1401 and provides information for the user display 1412. For one preferred embodiment, computation of SaO2 can proceed by using measurements from a subset of the sources and detectors having spacings similar to those of the forehead patches, and then using the time-varying component extraction method described with respect to FIGS. 12-13, supra. For another preferred embodiment, locations within the three-dimensional SctO2 volume known to be locationally associated with an arterial vessel are identified, and then the values for that location are used for the SaO2 determination. The identification can be based on a priori anatomical knowledge from other types of scans and/or from analysis of the dynamic behavior of the three-dimensional SctO2 volume or its underlying absorption coefficient volumes. Preferably, the user display 1412 simultaneously displays the following: a two- or three- dimensional color-coded map 1414 based on the three-dimensional volume of SctO2 levels, left and right regional SctO2 trend graphs, left and right regional SctO2 current levels, left and right SaO2 trend graphs, left and right SaO2 current levels, a heart pulse trend graph, a current pulse rate, an absolute [HbT] trend graph, and an absolute [HbT] current level.

As used hereinabove, chromophore refers to a substance in a physiological medium which exhibits at least minimum optical interaction with electromagnetic waves transmitting therethrough. Such chromophores may include solvents of a medium, solutes dissolved in the medium, and/or other substances included in such medium. Examples of such chromophores may include, but not limited to, oxygenated hemoglobin, deoxygenated hemoglobin, cytochromes, cytosomes, cytosols, enzymes, hormones, neurotransmitters, chemical or chemotransmitters, proteins, cholesterols, apoproteins, lipids, carbohydrates, blood cells, water, and other optical materials present in the animal or human cells, tissues or body fluid. Chromophores may also include extra-cellular substances which may be injected into the medium for therapeutic and/or imaging purposes or for creating interaction with electromagnetic waves. Typical examples of such chromophores may include, but not limited to, dyes, contrast agents, and other image-enhancing agents, each of which may be designed to exhibit optical interaction with electromagnetic waves having wavelengths in a specific range. Hemoglobin refer to oxygenated hemoglobin (i.e., HbO) and/or deoxygenated hemoglobin (i.e., Hb) or sum thereof. As used herein, “property” of a chromophore (or hemoglobin) may be an intensive property such as their concentrations, a sum of such concentrations, a difference therebetween, and a ratio thereof. Such property may also be extensive property such as, e.g., volume, mass, weight, volumetric flow rate, and mass flow rate of the chromophores (or hemoglobins).

Whereas many alterations and modifications of the preferred embodiments will no doubt become apparent to a person of ordinary skill in the art after having read the foregoing description, it is to be understood that the particular embodiments shown and described by way of illustration are in no way intended to be considered limiting. By way of example, although detection and volumetric imaging of blood oxygenation levels is described in several of the preferred embodiments above, the described methods have also been found useful for detection and volumetric imaging of carbon dioxide (CO₂) levels in the deep brain and elsewhere. In a similar way as O₂ delivery in biological system, CO₂ partially resolves or dissolves in the blood, binds with hemoglobin to form Hb_CO₂, and eventually is released in the lung during gas exchange. Hb_CO2 has a distinguished absorption coefficient and its concentration can be obtained through similar spectroscopic measurement and principles as the oxygenated hemoglobin determination described in Cheng, supra. Through measuring Hb_CO₂, the PH+ value can be derived as well as the CO₂ partial pressure inside cells. Therefore, reference to the details of the preferred embodiments are not intended to limit their scope, which is limited only by the scope of the claims set forth below.

What is claimed is:

1. A method for non-invasive monitoring of a biological volume, comprising:
causing each of a plurality of optical sources and a plurality of optical detectors to be positioned in optical coupling with the biological volume, each of the plurality of optical sources defining a source-detector pair with each of the plurality of optical detectors;
causing the plurality of optical sources to emit optical radiation into the biological volume at substantially non-overlapping time intervals, and causing each optical detector to detect optical radiation impinging thereupon from the biological volume, whereby at least one optical measurement is acquired for each source-detector pair;
for each source-detector pair, processing the at least one optical measurement to compute a requisite property value associated therewith, said processing including accounting for weighting a volumetric basis region corresponding to that source-detector pair by said requisite property value, said volumetric basis region being representative of an estimated subvolume of the biological volume encountered by optical radiation emitted from that source and propagating to that detector, and said processing further accounting for accumulating the weighted volumetric basis regions into a volumetric cumulative array; and
generating a display output based at least in part on said volumetric cumulative array.

2. The method of claim 1, wherein said processing comprises image reconstruction based on said optical measurements prior to said generation of said display output.

3. The method of claim 1, wherein the volumetric basis region associated with each of said source-detector pairs is predetermined by computer simulation of optical propagation between a like source and a like detector located at like positions on a model version of the biological volume.

4. The method of claim 3, wherein said volumetric basis region comprises voxels extending from said like source to like detector, each voxel being weighted according to a statistical probability of being encountered by photons emitted from that like source and propagating to that like detector.

5. The method of claim 1, wherein said requisite property value computed from said optical measurement comprises an absorption coefficient.

6. The method of claim 1, wherein said at least one optical measurement for each source-detector pair comprises a plurality of phase shift measurements for a respective plurality of intervals in which said optical source emits at a respective plurality of combinations of optical wavelength and intensity modulation frequency.

7. The method of claim 6, wherein said requisite property value computed from said optical measurement comprises a plurality of absorption coefficients, each absorption coefficient corresponding to one of said optical wavelengths emitted by said optical source.

8. The method of claim 7, wherein said generating the display output comprises:
for each voxel in said volumetric cumulative array, computing at least one chromophore concentration based on said plurality of absorption coefficients for that voxel; and
providing the display output based on said at least one computed chromophore concentrations.

9. The method of claim 8, wherein said biological volume comprises at least a portion of the human brain, wherein said at least one computed chromophore concentration comprises an oxygenated hemoglobin concentration [HbO] and a deoxygenated hemoglobin concentration [Hb], and wherein said providing the display output comprises:
for each voxel in said volumetric cumulative array, computing an oxygen saturation level SctO2 from said oxygenated and deoxygenated hemoglobin concentrations to result in a three-dimensional map of oxygen saturation levels; and
displaying at least one of a two-dimensional cross-section and a three-dimensional rendering of said three-dimensional oxygen saturation map on the display output.

10. The method of claim 1, wherein said biological volume comprises at least a portion of the human brain, wherein said requisite property comprises one of a scattering coefficient, an attenuation coefficient, an oxygenated hemoglobin concentration [HbO], a deoxygenated hemoglobin concentration [Hb], a total hemoglobin concentration [HbT], an oxygen saturation level, an Hb_CO₂ concentration, a PH+ value, and a CO₂ partial pressure value, and wherein said generating a display output comprises displaying at least one of a two-dimensional cross-section and a three-dimensional rendering of the volumetric cumulative array.

11. The method of claim 1, wherein said biological volume comprises at least a portion of the human brain, wherein said plurality of optical sources includes at least six (6) optical sources, and wherein said plurality of optical detectors includes at least six (6) optical detectors.

12. The method of claim 11, wherein said plurality of optical sources includes at least forty (40) optical sources, and wherein said plurality of optical detectors includes at least forty (40) optical detectors.

13. A method for facilitating non-invasive monitoring of a biological volume, comprising:
receiving first information representative of optical measurements acquired from a plurality of optical source-detector pairs positioned in optical coupling with the biological volume;
processing said first information to compute second information representative of a requisite property value for each source-detector pair; and
processing said second information to compute third information representative of a three-dimensional map of the requisite property value, wherein said processing said second information to compute said third information comprises:
receiving, for each optical source-detector pair, predetermined fourth information representative of a volumetric basis region corresponding to that source-detector pair, said volumetric basis region being representative of an estimated subvolume of the biological volume encountered by optical radiation emitted from that source and propagating to that detector;
for each optical source-detector pair, weighting the corresponding volumetric basis region according to the requisite property value for that source-detector pair; and
computing a voxelwise sum of all of said weighted volumetric basis regions to generate said third information representative of the three-dimensional map of the requisite property value.

14. The method of claim 13, further comprising:
filtering and normalizing said voxelwise sum; and
providing an output display based on said three-dimensional map of the requisite property value.

15. The method of claim 14, wherein said predetermined fourth information is precomputed for each of said optical source-detector pairs by computer simulation of optical propagation between a like source and a like detector located at like positions on a model version of the biological volume.

16. The method of claim 15, wherein said volumetric basis region comprises voxels extending from said like source to like detector, each voxel being weighted according to a statistical probability of being encountered by photons emitted from that like source and propagating to that like detector.

17. The method of claim 13, wherein said biological volume comprises at least a portion of the human brain, wherein said requisite property comprises one of a scattering coefficient, an attenuation coefficient, an oxygenated hemoglobin concentration [HbO], a deoxygenated hemoglobin concentration [Hb], a total hemoglobin concentration [HbT], an oxygen saturation level, an Hb_CO₂ concentration, a PH+ value, and a CO₂ partial pressure value, and wherein said providing an output display comprises displaying at least one of a two-dimensional cross-section and a three-dimensional rendering of said three-dimensional map.

18. A computer program product tangibly stored on a computer-readable medium for facilitating non-invasive monitoring of a biological volume, comprising:
computer code for receiving first information representative of optical measurements acquired from a plurality of optical source-detector pairs optically coupled with the biological volume;
computer code for processing said first information to compute second information representative of a requisite property value for each source-detector pair; and
computer code for processing said second information to compute third information representative of a three-dimensional map of the requisite property value, wherein said computer code for processing said second information to compute said third information comprises:
computer code for receiving, for each optical source-detector pair, predetermined fourth information representative of a volumetric basis region corresponding to that source-detector pair, said volumetric basis region being representative of an estimated subvolume of the biological volume encountered by optical radiation emitted from that source and propagating to that detector;
computer code for weighting, for each optical source-detector pair, the corresponding volumetric basis region according to the requisite property value for that source-detector pair; and
computer code for computing a voxelwise sum of all of said weighted volumetric basis regions to generate said third information representative of the three-dimensional map of the requisite property value.

19. The computer program product of claim 18, further comprising:
computer code for filtering and normalizing said voxelwise sum; and
computer code for providing an output display based on said three-dimensional map of the requisite property value.

20. The computer program product of claim 19, further comprising computer code for predetermining said volumetric basis region for each of said optical source-detector pairs by simulating optical propagation between a like source and a like detector located at like positions on a model version of the biological volume.

21. A computer program product tangibly stored on a computer-readable medium for facilitating non-invasive monitoring of a biological volume, comprising:
computer code for receiving first information representative of optical measurements acquired from a plurality of optical source-detector pairs optically coupled with the biological volume;
computer code for processing said first information to compute second information representative of a requisite property value for each source-detector pair;
computer code for processing said second information to compute third information representative of a three-dimensional map of the requisite property value, wherein said computer code for processing said second information to compute said third information comprises computer code for reconstructing an image representative of said three-dimensional map, said reconstructing accounting for volumetric basis regions related to the source-detector pairs, said volumetric basis regions being representative of estimated subvolumes of the biological volume encountered by optical radiation emitted from the sources and propagating to the detectors; and
computer code for further processing said third information to cause a selected two-dimensional or three-dimensional display of display information derived at least in part from said third information.

22. A computer program product as in claim 21, wherein the biological volume comprises a patient's brain.

23. The method of claim 1, wherein said requisite property value computed from said optical measurement comprises an attenuation coefficient.
